(19) United States
(12) Patent Application Publication
(10) Pub. No.: US 2005/0004010 A1
(43) Pub. Date: Jan. 6, 2005

(54) Cobalamin conjugates useful as imaging agents and as antitumor agents

(75) Inventors: Douglas A. Collins, Rochester, MN (US); Henricus P.C. Hogenkamp, Roseville, MN (US)

Correspondence Address: KING & SPALDING LLP, 191 PEACHTREE STREET, N.E., ATLANTA, GA, 30303-1763, US

(73) Assignees: Mayo Foundation For Medical Education; Research AND Regents of the University of Minnesota

(21) Appl. No.: 10/859,865

(22) Filed: Jun. 3, 2004

Related U.S. Application Data
(63) Continuation of application No. 09/690,198, filed on Oct. 16, 2000, now abandoned.
Application No. 09/690,198 is a continuation of application No. PCT/US00/10097, filed on Apr. 15, 2000.
(60) Provisional application No. 60/159,874, filed on Oct. 15, 1999.

(51) Int. Cl. A61K 38/14; C07K 9/00; A61K 31/714; C07H 23/00

(52) U.S. Cl. 514/7; 514/52; 536/26.4; 530/322

(57) ABSTRACT

The invention provides cobalamin derivatives which are useful for medical treatment and diagnosis.

### RELATED APPLICATION

The application claims priority to U.S. Provisional Application Ser. No. 60/159,874, filed 15 Oct. 1999.

### BACKGROUND OF THE INVENTION

Cancer is a general term frequently used to indicate any of the various types of malignant neoplasms (i.e., abnormal tissue that grows by cellular proliferation more rapidly than normal), most of which invade surrounding tissue, may metastasize to several sites, are likely to recur after attempted removal, and causes death unless adequately treated. Stedman's Medical Dictionary, 25th Edition Illustrated, Williams & Wilkins, 1990. Approximately 1.2 million Americans are diagnosed with cancer each year, 8,000 of which are children. In addition, 500,000 Americans die from cancer each year in the United States alone. Specifically, lung and prostate cancer are the top cancer killers for men while lung and breast cancer are the top cancer killers for women. It is estimated that cancer-related costs account for about 10 percent of the total amount spent on disease treatment in the United States. CNN Cancer Facts, http://www.cnn.comi/HEALTH/9511/conquer_cancer/facts/index.html, page 2 of 2, Jul. 18, 1999.

Although a variety of approaches to cancer therapy (e.g., surgical resection, radiation therapy, and chemotherapy) have been available and commonly used for many years, cancer remains one of the leading causes of death in the world. This is due in part to the therapies themselves causing significant toxic side-effects as well as the re-emergence of the deadly disease.

The toxicity associated with conventional cancer chemotherapy is due primarily to a lack of specificity of the chemotherapeutic agent. Unfortunately, anti-cancer drugs by themselves typically do not distinguish between malignant and normal cells. As a result, anti-cancer drugs are absorbed by both cell types. Thus, conventional chemotherapeutic agents not only destroy diseased cells, but also destroy normal, healthy cells. To overcome this limitation, therapeutic strategies that increase the specificity, increase the efficacy, as well as reduce the toxicity of anti-cancer drugs are being explored. One such strategy that is being aggressively pursued is drug targeting.

An objective of drug targeting is to deliver drugs to a specific site of action through a carrier system. Such targeting achieves at least two major aims of drug delivery. The first is to deliver the maximum dose of therapeutic agent to diseased cells. The second is the avoidance of uptake by normal, healthy cells. Thus, targeted drug delivery systems result in enhancing drug accumulation in tumors while decreasing exposure to susceptible healthy tissues. As such, the efficacy is increased while the toxicity is decreased.

Two classes of compounds with a propensity for localizing in malignant tumors are the porphyrins and the related phthalocyanines. The biochemical basis by which these compounds achieve elevated concentration in malignant tumors is unknown, but this observation has served as the rationale for the use of hematoporphyrin derivatives in the photodynamic therapy of cancer (Dougherty, T. J. et al., Porphyrin Photosensitization, 3-13, New York: Plenum Publishing Corp. (1981)).

For several years after the isolation of vitamin B₁₂ as cyanocobalamin in 1948, it was assumed that cyanocobalamin and possibly hydroxocobalamin, its photolytic breakdown product, occurred in man. Since then it has been recognized that cyanocobalamin is an artifact of the isolation of vitamin B₁₂ and that hydroxocobalamin and the two coenzyme forms, methylcobalamin and adenosylcobalanin, are the naturally occurring forms of the vitamin.

The structure of these various forms is shown in FIG. 1, wherein X is CN, OH, CH₃ or adenosyl, respectively. Hereinafter, the term cobalamin will be used to refer to all of the molecule except the X group. The fundamental ring system without cobalt (Co) or side chains is called corrin and the octadehydrocorrin is called corrole. FIG. 1 is adapted from The Merck Index, Merck & Co. (11th ed. 1989), wherein X is above the plane defined by the corrin ring and the nucleotide is below the plane of the ring. The corrin ring has attached seven amidoalkyl (H₂NC(O)Alk) substituents, at the 2, 3, 7, 8, 13, 18 and 23 positions, which can be designated a-g respectively. See D. L. Anton et al., J. Amer. Chem. Soc., 102, 2215 (1980). The 2, 3, 7, 8, and 13 positions are shown in FIG. 1 as positions a-e, respectively.

Cells undergoing rapid proliferation have been shown to have increased uptake of thymidine and methionine. (See, for example, M. E. van Eijkeren et al., Acta Oncologica, 31, 539 (1992); K. Kobota et al., J. Nucl. Med., 32, 2118 (1991) and K. Higashi et al., J. Nucl. Med., 34, 773 (1993)). Since methylcobalamin is directly involved with methionine synthesis and indirectly involved in the synthesis of thymidylate and DNA, it is not surprising that methylcobalamin as well as Cobalt-57-cyanocobalamin have also been shown to have increased uptake in rapidly dividing tissue (for example, see, B. A. Cooper et al., Nature, 191, 393 (1961); H. Flodh, Acta Radiol. Suppl., 284, 55 (1968); L. Bloomquist et al., Experientia, 25, 294 (1969)). Additionally, up-regulation in the number of transcobalamin II receptors has been demonstrated in several malignant cell lines during their accelerated thymidine incorporation and DNA synthesis (see, J. Lindemans et al., Exp. Cell. Res., 184, 449 (1989); T. Amagasaki et al., Blood, 26, 138 (1990) and J. A. Begly et al., J. Cell Physiol., 156, 43 (1993).

PCT Application WO 98/08859 discloses bioconjugates (i.e., conjugates containing a bioactive agent and an organo-cobalt complex in which the bioactive agent is covalently bound directly or indirectly, via a spacer, to the cobalt atom). The organo-cobalt complex can be cobalamin and the bioactive agent can be a chemotherapeutic agent. However, only one bioactive agent (i.e., chemotherapeutic agent) is attached to the organo-cobalt complex (i.e., cobalamin) and the attachment is to the cobalt atom (i.e., the 6-position of cobalamin). The bioactive agent is released from the bioconjugate by the cleavage of the weak covalent bond between the bioactive agent and the cobalt atom as a result of normal displacement by cellular nucleophiles or enzymatic action, or by application of an external signal (e.g., light, photoexcitation, ultrasound, or the presence of a magnetic filed).

Despite the above findings, there is currently a need for chemotherapeutic agents that have improved specificity (i.e., localize in tumor cells in high concentration compared to normal cells), or efficacy, and for chemotherapeutic agents which can selectively target cancer cells.

### SUMMARY OF THE INVENTION

Applicant has discovered cobalamin conjugates (i.e., conjugates of Vitamin B₁₂ and a chemotherapeutic agent) that are useful to treat and/or image tumors. The cobalamin conjugates have a low toxicity, a high activity against diseased cells, and a high specificity (i.e., they localize in tumor cells in a higher concentration than in normal cells).

The present invention provides a compound (i.e., cobalamin conjugate of the present invention) wherein a residue of a compound of formula I (FIG. 1) is linked directly or by a linker to a residue of one or more chemotherapeutic agents; wherein X is CN, OH, CH₃, or adenosyl; or a pharmaceutically acceptable salt thereof.

The present invention also provides a compound (i.e., a cobalamin conjugate of the present invention) wherein a residue of a compound of formula I (FIG. 1) is linked directly or by a linker to a residue of a chemotherapeutic agent through the 6-position and wherein a residue of the compound of formula I is linked directly or by a linker to a residue of one or more additional chemotherapeutic agents; or a pharmaceutically acceptable salt thereof.

The present invention also provides a compound (i.e., cobalamin conjugate of the present invention) of formula II 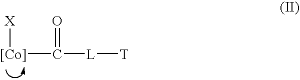
wherein 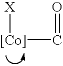
is a residue of the compound of formula I; X is CN, OH, CH₃, adenosyl, or LL-TT wherein LL is a linker or is absent and TT is a residue of a chemotherapeutic agent; L is a linker or absent; and T is a residue of a chemotherapeutic agent; or a pharmaceutically acceptable salt thereof.

The present invention also provides a compound (i.e., cobalamin conjugate of the present invention) of formula II 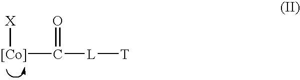
wherein 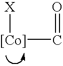
is a residue of the compound of formula I; X is LL-TT wherein LL is a linker or is absent and TT is a residue of a chemotherapeutic agent; L is a linker or absent; and T is a residue of a chemotherapeutic agent; or a pharmaceutically acceptable salt thereof.

The present invention also provides a compound (i.e., cobalamin conjugate of the present invention) of formula III: 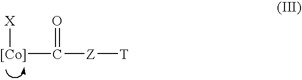
wherein X is CN, OH, CH₃, adenosyl, or ZZ-TT wherein ZZ is a linker or is absent and TT is a residue of a chemotherapeutic agent; Z is —N(R)—, —O—, —S—, or absent wherein R is H or (C₁-C₆)alkyl; and T is a residue of a chemotherapeutic agent; or a pharmaceutically acceptable salt thereof.

The present invention also provides a compound (i.e., cobalamin conjugate of the present invention) of formula III: 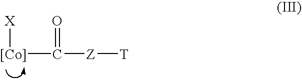
wherein X is LL-TT wherein LL is a linker or is absent and TT is a residue of a chemotherapeutic agent; Z is —N(R)—, —O—, —S—, or absent, wherein R is H or (C₁-C₆)alkyl; and T is a residue of a chemotherapeutic agent; or a pharmaceutically acceptable salt thereof.

The present invention also provides a compound wherein a residue of a compound of formula I (FIG. 1) is linked directly or by a linker to a residue of one or more chemotherapeutic agents; wherein X is CN, OH, CH₃, or adenosyl; wherein the compound of formula I is also linked directly or by a linker to a detectable radionuclide; or a pharmaceutically acceptable salt thereof.

The present invention also provides a pharmaceutical composition comprising a cobalamin conjugate of the present invention, or a pharmaceutically acceptable salt thereof; and a pharmaceutically acceptable carrier.

The present invention also provides a method of treating a tumor in a mammal in need of such treatment comprising administering to the mammal an effective amount of a cobalamin conjugate of the present invention, or a pharmaceutically acceptable salt thereof; and a pharmaceutically acceptable carrier.

The invention also provides a method for imaging a tumor in a mammal in need of such imaging comprising administering to the mammal a detectable amount of a cobalamin conjugate of the present invention; and detecting the presence of the compound.

The invention also provides a compound of the present invention for use in medical therapy or diagnosis.

The invention also provides the use of a compound of the present invention for the manufacture of a medicament for imaging a tumor in a mammal (e.g., a human).

The invention also provides the use of a compound of the present invention for the manufacture of a medicament for treating a tumor in a mammal (e.g., a human).

The invention also provides intermediates disclosed herein that are useful in the preparation of the compounds of the present invention as well as synthetic methods useful for preparing the compounds of the invention.

The cobalamin conjugate of the present invention has several characteristics which make it an attractive in vivo targeting agent. Vitamin B₁₂ is water soluble, has no known toxicity, and in excess is excreted by glomerular filtration. In addition, the uptake of vitamin B₁₂ can potentially be manipulated by the administration of nitrous oxide and other pharmacological agents (D. Swanson et al., Pharmaceuticals in Medical Imaging, MacMillan Pub. Co., NY (1990) at pages 621-628).

### BRIEF DESCRIPTION OF THE FIGURES

FIG. 1 illustrates a compound of formula I, wherein X is CN, OH, CH₃, adenosyl or a residue of a chemotherapeutic agent. The compound of formula I can be cyanocobalamin (X is CN), hydroxocobalamin (X is OH), methylcobalamin (X is CH₃), or adenosylcobalamin (X is adenosyl). In addition, the compound of formula I can be a cobalamin conjugate (X is a residue of a chemotherapeutic agent or X is a linker linked to a residue of a chemotherapeutic agent).

FIG. 2 illustrates a proposed synthesis of a compound wherein a residue of a compound of formula I is linked to linker, which is linked to a residue of a chemotherapeutic agent.

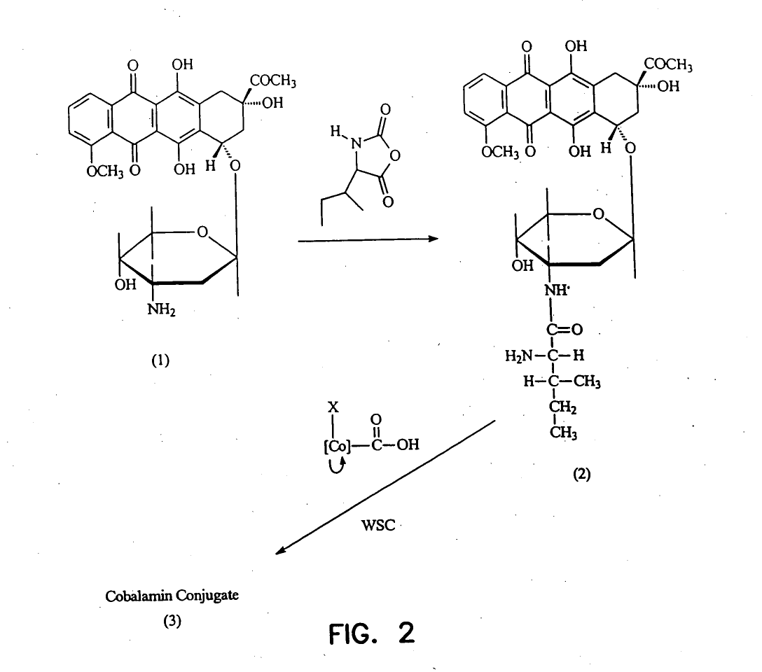

### DETAILED DESCRIPTION OF THE INVENTION

The following definitions are used, unless otherwise described: halo is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic.

Specific and preferred values listed below for radicals, substituents, and ranges, are for illustration only; they do not exclude other defined values or other values within defined ranges for the radicals and substituents.

It is appreciated that those skilled in the art will recognize that compounds of the present invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine antitumor activity using the standard tests described herein, or using other similar tests which are well known in the art.

Specifically, (C₁-C₆)alkyl can be methyl, ethyl, propyl, isopropyl, butyl, iso-butyl, sec-butyl, pentyl, 3-pentyl, or hexyl.

Specifically, (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl.

Specifically, (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl.

Specifically “aryl” can be phenyl, indenyl, or naphthyl.

Specifically (C₃-C₈)cycloalkyl can be cyclopropyl, cyclobutyl, cyclcopentyl, cyclohexyl, cycloheptyl or cyclooctyl.

As used herein, “adenosyl” is an adenosine radical in which any synthetically feasible atom or group of atoms have been removed, thereby providing an open valence. Synthetically feasible atoms which may be removed include the hydrogen atom of the hydroxy group at the 5′ position. Accordingly, adenosyl can conveniently be attached to the 6-position (i.e., the position occupied by X in the compound of formula I) of a compound of formula I via the 5′ position of adenosyl.

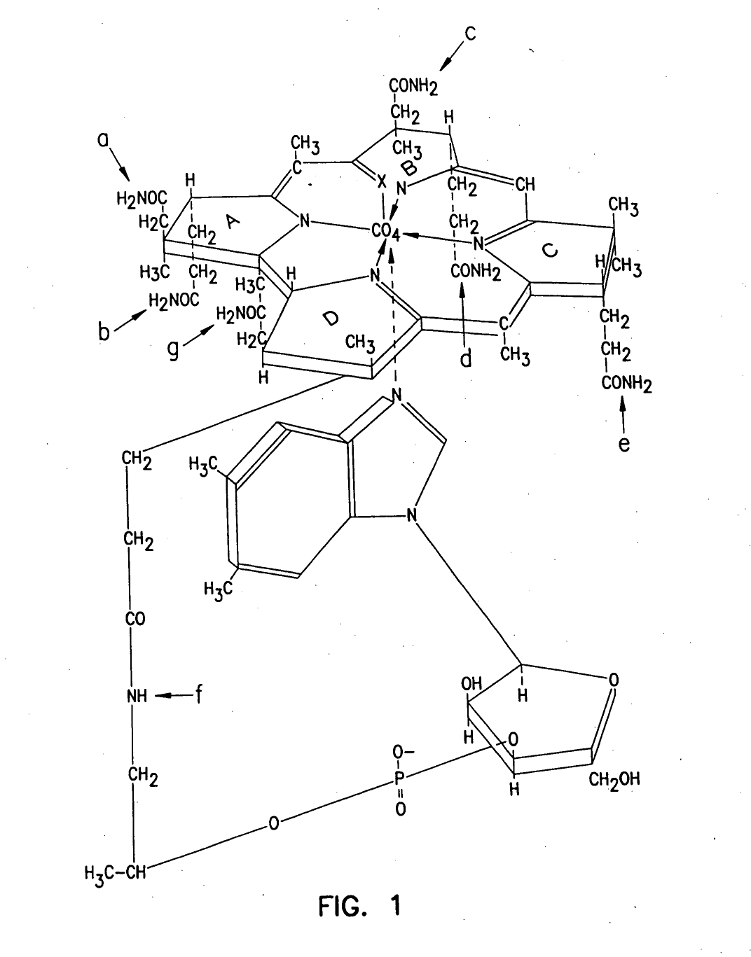

As used herein, a “residue of a compound of formula I” is a radical of a compound of formula I having an open valence. Any synthetically feasible atom or atoms of the compound of formula I can be removed to provide the open valence, provided the resulting compound is able to localize in or near a tumor. Based on the linkage that is desired, one skilled in the art can select suitably functionalized starting materials that can be derived from a compound of formula I using procedures that are known in the art. For example, suitable atoms that can be removed include the NH₂ group of the a-carboxamide (illustrated in FIG. 1), the NH₂ group of the b-carboxamide (illustrated in FIG. 1), the NH₂ group of the d-carboxamide (illustrated in FIG. 1), the NH₂ group of the e carboxamide (illustrated in FIG. 1), the hydrogen atom of the hydroxy group at the 3′ position of the sugar, and the hydrogen atom of the CH₂OH group at the 5′ position of the sugar ring may be removed. In addition, X at the 6-position (illustrated in FIG. 1) can be removed to provide an open valence to link a first chemotherapeutic agent.

As used herein, a “residue of a chemotherapeutic agent” is a radical of a chemotherapeutic agent having an open valence. Any synthetically feasible atom or atoms of the chemotherapeutic agent may be removed to provide the open valence, provided the bioactivity of the agent is retained when administered as a conjugate of the invention. In addition, the residue of the chemotherapeutic agent does not comprise a radionuclide. Based on the linkage that is desired, one skilled in the art can select suitably functionalized starting materials that can be derived from a chemotherapeutic agent using procedures that are known in the art.

As used herein, a “residue of doxorubicin or paclitaxel” is a radical of doxorubicin or a radical of paclitaxel having an open valence formed by removing a substituent (i.e., atom or group of atoms) from doxorubicin or by removing a substituent (i.e., atom or group of atoms) from paclitaxel. Any synthetically feasible atom or atoms of doxorubicin or paclitaxel may be removed to provide the open valence, provided useful bioactivity is retained when administered as a conjugate of the invention. Based on the linkage that is desired, one skilled in the art can select suitably functionalized starting materials that can be derived from doxorubicin or paclitaxel using procedures that are known in the art.

As used herein, an “amino acid” is a natural amino acid residue (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acid (e.g. phosphoserine; phosphothreonine; phosphotyrosine; hydroxyproline; gamma-carboxyglutamate; hippuric acid; octahydroindole-2-carboxylic acid; statine; 1,2,3,4,-tetrahydroisoquinoline-3-carboxylic acid; penicillamine; ornithine; citruline; α-methyl-alanine; para-benzoylphenylalanine; phenylglycine; propargylglycine; sarcosine; and tert-butylglycine) residue having one or more open valences. The term also comprises natural and unnatural amino acids bearing amino protecting groups (e.g. acetyl, acyl, trifluoroacetyl, or benzyloxycarbonyl), as well as natural and unnatural amino acids protected at carboxy with protecting groups (e.g. as a (C₁-C₆)alkyl, phenyl or benzyl ester or amide). Other suitable amino and carboxy protecting groups are known to those skilled in the art (See for example, T. W. Greene, Protecting Groups In Organic Synthesis; Wiley: New York, 1981; D. Voet, Biochemistry, Wiley: New York, 1990; L. Stryer, Biochemistry, (3rd Ed.), W. H. Freeman and Co.: New York, 1975; J. March, Advanced Organic Chemistry Reactions, Mechanisms and Structure, (2nd Ed.), McGraw Hill: New York, 1977; F. Carey and R. Sundberg, Advanced Organic Chemistry Part B: Reactions and Synthesis, (2nd Ed.), Plenum: New York, 1977; and references cited therein). According to the invention, the amino or carboxy protecting group can comprise a radionuclide (e.g., Fluorine-18, Iodine-123, or Iodine-124).

As used herein, a “peptide” is a sequence of 2 to 25 amino acids (e.g. as defined herein) or peptidic residues. The sequence may be linear or cyclic. For example, a cyclic peptide can be prepared or may result from the formation of disulfide bridges between two cysteine residues in a sequence. A peptide can be linked through the carboxy terminus, the amino terminus, or through any other convenient point of attachment, such as, for example, through the sulfur of a cysteine. Specifically, a peptide comprises 2 to about 20, 2 to about 15, or 2 to about 12 amino acids. Peptide derivatives can be prepared as disclosed in U.S. Pat. Nos. 4,612,302; 4,853,371; and 4,684,620, or as described in the Examples herein. Peptide sequences specifically recited herein are written with the amino terminus on the left and the carboxy terminus on the right.

Specifically, the peptide can be poly-L-lysine, poly-L-glutamic acid, poly-L-aspartic acid, poly-L-histidine, poly-L-ornithine, poly-L-serine, poly-L-threonine, poly-L-tyrosine, poly-L-lysine-L-phenylalanine or poly-L-lysine-L-tyrosine.

Chemotherapeutic Agent

As used herein, a “chemotherapeutic agent” is a compound that has biological activity against one or more forms of cancer and can be linked to the residue of a compound of formula I without losing its anticancer activity. Suitable chemotherapeutic agents include antineoplasts. Representative antineoplasts include adjuncts, androgen inhibitors, antibiotic derivatives, antiestrogens, antimetabolites, cytotoxic agents, hormones, immunomodulators, nitrogen mustard derivatives and steroids. Physicians' Desk Reference, 50th Edition, 1996.

Representative adjuncts include levamisole, gallium nitrate, granisetron, sargramostim strontium-89 chloride, filgrastim, pilocarpine, dexrazoxane, and ondansetron. Physicians' Desk Reference, 50th Edition, 1996.

Representative androgen inhibitors include flutamide and leuprolide acetate. Physicians' Desk Reference, 50th Edition, 1996.

Representative antibiotic derivatives include doxorubicin, bleomycin sulfate, daunorubicin, dactinomycin, and idarubicin.

Representative antiestrogens include tamoxifen citrate and analogs thereof. Physicians' Desk Reference, 50th Edition, 1996. Additional antiestrogens include nonsteroidal antiestrogens such as toremifene, droloxifene and roloxifene. Magarian et al., Current Medicinal Chemistry, 1994, Vol. 1, No. 1.

Representative antimetabolites include fluorouracil, fludarabine phosphate, floxuridine, interferon alfa-2b recombinant, methotrexate sodium, plicamycin, mercaptopurine, and thioguanine. Physicians' Desk Reference, 50th Edition, 1996.

Representative cytotoxic agents include doxorubicin, carmustine [BCNU], lomustine [CCNU], cytarabine USP, cyclophosphamide, estramucine phosphate sodium, altretamine, hydroxyurea, ifosfamide, procarbazine, mitomycin, busulfan, cyclophosphamide, mitoxantrone, carboplati, cisplati, cisplatin, interferon alfa-2a recombinant, paclitaxel, teniposide, and streptozoci. Physicians' Desk Reference, 50th Edition, 1996.

Representative hormones include medroxyprogesterone acetate, estradiol, megestrol acetate, octreotide acetate, diethylstilbestrol diphosphate, testolactone, and goserelin acetate. Physicians' Desk Reference, 50th Edition, 1996.

Representative immunodilators include aldesleukin. Physicians' Desk Reference, 50th Edition, 1996.

Representative nitrogen mustard derivatives include melphalan, chlorambucil, mechlorethamine, and thiotepa. Physicians' Desk Reference, 50th Edition, 1996.

Representative steroids include betamethasone sodium phosphate and betamethasone acetate. Physicians' Desk Reference, 50th Edition, 1996.

Specifically, the chemotherapeutic agent can be an antineoplastic agent.

Specifically, the antineoplastic agent can be a cytotoxic agent.

Specifically, the cytotoxic agent can be paclitaxel or doxorubicin.

Additional suitable chemotherapeutic agents include alkylating agents, antimitotic agents, plant alkaloids, biologicals, topoisomerase I inhibitors, topoisomerase II inhibitors, and synthetics. AntiCancer Agents by Mechanism, http://www.dtp.nci.nih.gov/docs/cancer/searches/standard_mechanism_list.html, Apr. 12, 1999; Approved Anti-Cancer Agents, http://www.ctep.info.nih.gov/handbook/HandBookText/fda_agen.htm, pages 1-7, Jun. 18, 1999; MCMP 611 Chemotherapeutic Drugs to Know, http//www.vet.purdue.edu/depts/bms/courses/mcmp611/chrx/drg2no61.html, Jun. 24, 1999; and Chemotherapy, http://www.vetmed.lsu.edu/oncology/Chemotherapy.htm, Apr. 12, 1999.

Representative alkylating agents include asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxo-rubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864. AntiCancer Agents by Mechanism, http://dtp.nci.nih.gov/docs/cancer/searches/standard_mechanism_list.html, Apr. 12, 1999.

Representative antimitotic agents include allocolchicine, Halichondrin B, colchicine, colchicine derivatives, dolastatin 10, maytansine, rhizoxin, paclitaxel derivatives, paclitaxel, thiocolchicine, trityl cysteine, vinblastine sulfate, and vincristine sulfate. AntiCancer Agents by Mechanism, http://dtp.nci.nih.gov/docs/cancer/searches/standard_mechanism_list.html, Apr. 12, 1999.

Representative plant alkaloids include actinomycin D, bleomycin, L-asparaginase, idarubicin, vinblastine sulfate, vincristine sulfate, mitramycin, mitomycin, daunorubicin, VP-16-213, VM-26, navelbine and taxotere. Approved Anti-Cancer Agents, http://ctep.info.nih.gov/handbook/HandBookText/fda agent.htm, Jun. 18, 1999.

Representative biologicals include alpha interferon, BCG, G-CSF, GM-CSF, and interleukin-2. Approved Anti-Cancer Agents, http://ctep.info.nih.gov/handbook/HandBookText/fda_agent.htm, Jun. 18, 1999.

Representative topoisomerase I inhibitors include camptothecin, camptothecin derivatives, and morpholinodoxorubicin. AntiCancer Agents by Mechanism, http://dtp.nci.nih.gov/docs/cancer/searches/standard_mechanism_list.html, Apr. 12, 1999.

Representative topoisomerase II inhibitors include mitoxantron, amonafide, m-AMSA, anthrapyrazole derivatives, pyrazoloacridine, bisantrene HCl, daunorubicin, deoxydoxorubicin, menogaril, N,N-dibenzyl daunomycin, oxanthrazole, rubidazone, VM-26 and VP-16. AntiCancer Agents by Mechanism, http://dtp.nci.nih.gov/docs/cancer/searches/standard_mechanism_list.html, Apr. 12, 1999.

Representative synthetics include hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium. Approved Anti-Cancer Agents, http://ctep.info.nih.gov/handbook/HandBookText/fda agen.htm, Jun. 18, 1999.

Compound of Formula I/Chemotherapeutic Agent Linkage

The residue of a chemotherapeutic agent can be directly linked to the residue of a compound of formula I through an amide (e.g., —N(R)C(═O)— or —C(═O)N(R)—), ester (e.g., —OC(═O)— or —C(═O)O—), ether (e.g., —O—), amino (e.g., —N(R)—), ketone (e.g., —C(═O)—), thioether (e.g., —S—), sulfinyl (e.g., —S(O)—), sulfonyl (e.g., —S(O)₂—), or a direct (e.g., C—C bond) linkage, wherein each R is independently H or (C₁-C₆)alkyl. Such a linkage can be formed from suitably functionalized starting materials using synthetic procedures that are known in the art. Based on the linkage that is desired, one skilled in the art can select suitably functional starting materials that can be derived from a residue of a compound of formula I and from a given residue of a chemotherapeutic agent using procedures that are known in the art.

The residue of the chemotherapeutic agent can be directly linked to any synthetically feasible position on the residue of a compound of formula I, provided if a residue of a chemotherapeutic agent is attached to a residue of a compound of formula I at the 6-position, the residue of a compound of formula I is attached to a residue of another chemotherapeutic agent or to a detectable radionuclide. Suitable points of attachment include, for example, the b-carboxamide, the d-carboxamide, and the e-carboxamide (illustrated in FIG. 1), as well as the 6-position (the position occupied by X in FIG. 1), and the 5′-hydroxy and the 3′-hydroxy groups on the 5-membered sugar ring, although other points of attachment are possible. U.S. Pat. No. 5,739,313 discloses compounds (e.g., cyanocobalamin-b-(4-aminobutyl)amide, methylcobalamin-b-(4-aminobutyl)amide, and adenosylcobalamin-b-(4-aminobutyl)amide) that are useful intermediates for the preparation of compounds of the present invention.

Compounds wherein the residue of a chemotherapeutic agent is directly linked to the 6-position of a compound of formula I can be prepared by reducing a corresponding Co (II) compound of formula I to form a nucleophilic Co (I) compound and treating this Co (I) compound with a residue of a chemotherapeutic agent (or a derivative thereof) comprising a suitable leaving group, such as a halide (e.g., a chloride).

The invention also provides compounds having more than one chemotherapeutic agent directly attached to a compound of formula I. For example, the residue of a chemotherapeutic agent can be directly linked to a residue of the b-carboxamide of the compound of formula I and a residue of another chemotherapeutic agent can be directly linked to a residue of the d-carboxamide of the compound of formula I. In addition, the residue of a chemotherapeutic agent can be directly linked to the 6-position of the compound of formula I and a residue of another chemotherapeutic agent can be directly linked to a residue of the b-, d- or e-carboxamide of the compound of formula I.

In addition to being directly linked to the residue of a compound of formula I, the residue of a chemotherapeutic agent can also be linked to the residue of a compound of formula I by a suitable linker. The structure of the linker is not crucial, provided it yields a compound of the invention which has an effective therapeutic index against the target cells, and which will localize in or near tumor molecules, which properties can be determined by those skilled in the art with assays that are known in the art.

Suitable linkers include linkers that separate the residue of a compound of formula I and the chemotherapeutic agent by about 5 angstroms to about 200 angstroms, inclusive, in length. Other suitable linkers include linkers that separate the residue of a compound of formula I and the chemotherapeutic agent by about 5 angstroms to about 100 angstroms, as well as linkers that separate the residue of a compound of formula I and the chemotherapeutic agent by about 5 angstroms to about 50 angstroms, or by about 5 angstroms to about 25 angstroms. Suitable linkers are disclosed, for example, in U.S. Pat. No. 5,735,313.

Specifically, the linker can be a divalent radical of the formula W-A-Q wherein A is (C₁-C₆)alkyl, (C₂-C₆)alkenyl, (C₂-C₆)alkynyl, (C₃-C₈)cyclo-alkyl, or (C₆-C₁₀)aryl, wherein W and Q are each independently —N(R)C(═O)—, —C(═O)N(R)—, —OC(═O)—, —C(═O)O—, —O—, —S—, —S(O)—, —S(O)₂—, —N(R)—, —C(═O)—, or a direct bond; wherein each R is independently H or (C₁-C₆)alkyl.

Specifically, the linker can be a divalent radical, i.e., 1,-divalent radicals formed from a peptide or an amino acid. The peptide can comprise 2 to about 20 amino acids, 2 to about 15 amino acids, or 2 to about 12 amino acids. The peptide or amino acid can optionally be protected, as described herein.

Specifically, the peptide can be poly-L-lysine (i.e., [—NHCH—[(CH₂)₄NH₂]CO-]ₘ-Q, wherein Q is H, (C₁-C₁₄)alkyl, or a suitable carboxy protecting group; and wherein m is about 2 to about 20). Specifically, poly-L-lysine contains about 5 to about 15 residues (i.e., m is between about 5 and about 15). More specifically, poly-L-lysine contains about 8 to about 11 residues (i.e., m is between about 8 and about 11).

Specifically, the peptide can be poly-L-glutamic acid, poly-L-aspartic acid, poly-L-histidine, poly-L-ornithine, poly-L-serine, poly-L-threonine, poly-L-tyrosine, poly-L-lysine-L-phenylalanine or poly-L-lysine-L-tyrosine.

Specifically, the linker can be prepared from 1,6-diaminohexane H₂N(CH₂)₆NH₂, 1,5-diaminopentane H₂N(CH₂)₅NH₂, 1,4-diaminobutane H₂N(CH₂)₄NH₂, or 1,3-diaminopropane H₂N(CH₂)₃NH₂.

Compound of Formula I/Linker/Chemotherapeutic Agent Linkage

The linker can be linked to (1) the residue of a chemotherapeutic agent and/or (2) the residue of a compound of formula I through an amide (e.g., —N(R)C(═O)— or —C(═O)N(R)—), ester (e.g., —OC(═O)— or —C(═O)O—), ether (e.g., —O—), amino (e.g., —N(R)—), ketone (e.g., —C(═O)—), thioether (e.g., —S—), sulfinyl (e.g., —S(O)—), sulfonyl (e.g., —S(O)₂—), or a direct (e.g., C—C bond) linkage, wherein each R is independently H or (C₁-C₆)alkyl. Such a linkage can be formed from suitably functionalized starting materials using synthetic procedures that are known in the art. Based on the linkage that is desired, one skilled in the art can select suitably functional starting materials that can be derived from a residue of a compound of formula I and from a given residue of a chemo-therapeutic agent using procedures that are known in the art.

The linker can be linked to any synthetically feasible position on the residue of a compound of formula I, provided if a linker is attached to a residue of a compound of formula I at the 6-position, at least one residue of a chemotherapeutic agent is linked directly or by a linker to a residue of the compound of formula I at a position other than the 6-position (i.e., the position occupied by X in the compound of formula I). Suitable points of attachment include, for example, a residue of the b-carboxamide, a residue of the d-carboxamide, and a residue of the e-carboxamide, the 6-position, as well as a residue of the 5′-hydroxy group and a residue of the 3′-hydroxy group on the 5-membered sugar ring, although other points of attachment are possible.

Compounds wherein the linker is linked to the 6-position of a compound of formula I can be prepared by preparing a nucleophilic Co (I) species as described herein above, and reacting it with a linker comprising a suitable leaving group, such as a halide (e.g. a chloride).

The invention also provides compounds having more than one chemotherapeutic agent attached to a compound of formula I, each through a linker. For example, the residue of a chemotherapeutic agent can conveniently be linked, through a linker, to a residue of the b-carboxamide of the compound of formula I and a residue of another chemotherapeutic agent can conveniently be linked, through a linker, to a residue of the d- or e-carboxamide of the compound of formula I. In addition, the residue of a chemotherapeutic agent can conveniently be linked, through a linker, to the 6-position of the compound of formula I and a residue of another chemotherapeutic agent can conveniently be linked, through a linker, to a residue of the b-, d- or e-carboxamide of the compound of formula I.

The invention also provides compounds having more than one chemotherapeutic agent attached to a compound of formula I, either directly or through a linker. For example, the residue of a chemotherapeutic agent can conveniently be linked, either directly or through a linker, to a residue of the b-carboxamide of the compound of formula I and a residue of another chemotherapeutic agent can conveniently be linked, either directly or through a linker, to a residue of the d- or e-carboxamide of the compound of formula I. In addition, the residue of a chemotherapeutic agent can conveniently be linked, either directly or through a linker, to the 6-position of the compound of formula I and a residue of another chemotherapeutic agent can conveniently be linked, either directly or through a linker, to a residue of the b-, d- or e-carboxamide of the compound of formula I.

Applicant has also discovered that it is possible to prepare a compound that is useful for both imaging and for treating tumors by incorporating one or more chemotherapeutic agents into a compound that also comprises one or more detectable radionuclides. Accordingly, the invention provides a residue of a compound of formula I which is linked to one or more residues of a chemotherapeutic agent; and which is also linked, directly or by a linker, to one or more detectable chelating groups including one or more detectable radionuclides.

Compound of Formula I/Linker/Detectable Chelating Group Linkage

The detectable chelating group can be linked to a residue of the compound of formula I by a linker. Suitable linkers are described herein. In addition, suitable points of attachment of the compound of formula I for the linker including the detectable chelating group are described herein.

A detectable chelating group including a radionuclide can be linked, via a linker, to a residue of a compound of the formula I. The linker can be linked to any synthetically feasible position on the residue of a residue of a compound of formula I; provided the compound localizes in or near tumors. Suitable points of attachment include, for example, a residue of the b-carboxamide, a residue of the d-carboxamide, and a residue of the e-carboxamide, the 6-position, as well as a residue of the 5′-hydroxy group and a residue of the 3′-hydroxy group on the 5-membered sugar ring, although other points of attachment are possible.

The invention also provides compounds having more than one detectable chelating group attached to a compound of formula I, each through a linker. For example, the detectable chelating group can conveniently be linked, through a linker, to a residue of the b-carboxamide of the compound of formula I and another detectable chelating group can conveniently be linked, through a linker, to a residue of the d- or e-carboxamide of the compound of formula I. In addition, the detectable chelating group can conveniently be linked, through a linker, to the 6-position of the compound of formula I and another detectable chelating group can conveniently be linked, through a linker, to a residue of the b-, d- or e-carboxamide of the compound of formula I.

The invention also provides compounds having more than one detectable radionuclide attached to a residue of the compound of formula I, either directly or through a linker.

Detectable Chelating Group

A “detectable chelating group” is a chelating group comprising a metallic radionuclide (e.g., a metallic radioisotope) capable of being detected in a diagnostic procedure in vivo or in vitro. Any suitable chelating group can be employed. Suitable chelating groups include those disclosed in U.S. Pat. No. 5,739,313. Specifically, the chelating group can be NTA, HEDTA, DCTA, RP414, MDP, DOTATOC, CDTA, HYNIC, EDTA, DTPA, TETA, DOTA, DOTMP, DCTA, 15N4, 9N3, 12N3, or MAG3 (or another suitable polyamino acid chelator), which are described herein below, or a phosphonate chelator (e.g. EDMT). More specifically, the chelating group can be. DTPA.

DTPA is diethylenetriaminepentaacetic acid; TETA is 1,4,8,11-tetraazacyclotetradecane-N,N′,N″,N′″-tetraacetic acid; DOTA is 1,4,7,10-tetraazacyclododecane-N,N′,N″,N′″-tetraacetic acid; 15N4 is 1,4,8,12-tetraazacyclopentadecane-N,N′,N″,N′″-tetraacetic acid; 9N3 is 1,4,7-triazacyclononane-N,N′,N″-triacetic acid; 12N3 is 1,5,9-triazacyclododecane-N,N′,N″-triacetic acid; MAG3 is (N-[N-[N-[(benzoylthio)acetyl]glycyl]glycyl]-glycine); and DCTA is a cyclohexane-based metal chelator of the formula 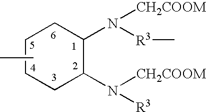
wherein R³ may by (C₁-C₄)alkyl or CH₂CO₂—, which may be attached through positions 4 or 5, or through the group R³ and which carries from 1 to 4 detectable metal or nonmetal cations (M), monovalent cations, or the alkaline earth metals. Thus, with metals of oxidation state+1, each individual cyclohexane-based molecule may carry up to 4 metal cations (where both R³ groups are CH₂COOM). As is more likely, with higher oxidation states, the number of metals will decrease to 2 or even 1 per cyclohexane skeleton. This formula is not intended to limit the molecule to any specific stereochemistry.

NTA, HEDTA, and DCTA are disclosed in Poster Sessions, Proceedings of the 46th Annual Meeting, J. Nuc. Med., p. 316, No. 1386. RP414 is disclosed in Scientific Papers, Proceedings of the 46th Annual Meeting, J. Nuc. Med., p. 123, No. 499. MDP is disclosed in Scientific Papers, Proceedings of the 46th Annual Meeting, J. Nuc. Med., p. 102, No. 413. DOTATOC is disclosed in Scientific Papers, Proceedings of the 46th Annual Meeting, J. Nuc. Med., p. 102, No. 414 and Scientific Papers, Proceedings of the 46th Annual Meeting, J. Nuc. Med., p. 103, No. 415. CDTA is disclosed in Poster Sessions, Proceedings of the 46th Annual Meeting, J. Nuc. Med., p. 318, No. 1396. HYNIC is disclosed in Poster Sessions, Proceedings of the 46th Annual Meeting, J. Nuc. Med., p. 319, No. 1398.

Bifunctional chelators (i.e., chelating groups) based on macrocyclic ligands in which conjugation is via an activated arm attached to the carbon backbone of the ligand can also be employed as a chelating group, as described by M. Moi et al., J. Amer. Chem., Soc., 49, 2639 (1989) (2-p-nitro-benzyl-1,4,7,10-tetraazacyclododecane-N,N′,N″,N′″-tetraacetic acid); S. V. Deshpande et al., J. Nucl. Med., 31, 473 (1990); G. Kuser et al., Bioconj. Chem., 1, 345 (1990); C. J. Broan et al., J. C. S. Chem. Comm., 23, 1739 (1990); and C. J. Anderson et al., J. Nucl. Med. 36, 850 (1995) (6-bromoacetamido-benzyl-1,4,8,11-tetraazacyclotetadecane-N,N′,N″,N′″-tetraacetic acid (BAT)).

In addition, the diagnostic chelator or diagnostic chelating groups can be any of the chelating groups disclosed in Scientific Papers, Proceedings of the 46th Annual Meeting, J. Nuc. Med., Wednesday, Jun. 9, 1999, p. 124, No. 500.

Specifically, the chelating group can be any one of the carbonyl complexes disclosed in Waibel et al., Nature Biotechnology, 897-901, Vol. 17, September 1999; or Sattelberger et al., Nature Biotechnology, 849-850, Vol. 17, September 1999.

Specifically, the detectable chelating group can be any of the carbonyl complexes disclosed in Waibel et al., Nature Biotechnology, 897-901, Vol. 17, September 1999; or Sattelberger et al., Nature Biotechnology, 849-850, Vol. 17, September 1999, further comprising a metallic radionuclide. More specifically, the detectable chelating group can be any of the carbonyl complexes disclosed in Waibel et al., Nature Biotechnology, 897-901, Vol. 17, September 1999; or Sattelberger et al., Nature Biotechnology, 849-850, Vol. 17, September 1999, further comprising Technetium-99m.

Specifically, the detectable chelating group can be any of the carbonyl complexes disclosed in Waibel et al., Nature Biotechnology, 897-901, Vol. 17, September 1999; or Sattelberger et al., Nature Biotechnology, 849-850, Vol. 17, September 1999, further comprising a metallic radionuclide. More specifically, the detectable chelating group can be any of the carbonyl complexes disclosed in Waibel et al., Nature Biotechnology, 897-901, Vol. 17, September 1999; or Sattelberger et al., Nature Biotechnology, 849-850, Vol. 17, September 1999, further comprising Rhenium-186 or Rhenium-188.

As used herein, a “detectable radionuclide” is any suitable radionuclide (i.e., radioisotope) capable of being detected in a diagnostic procedure in vivo or in vitro. Suitable detectable radionuclides include metallic radionuclides (i.e., metallic radioisotopes) and non-metallic radionuclides (i.e., non-metallic radioisotopes).

Metallic Radionuclides

Suitable metallic radionuclides (i.e., metallic radioisotopes or metallic paramagnetic ions) include Antimony-124, Antimony-125, Arsenic-74, Barium-103, Barium-140, Beryllium-7, Bismuth-206, Bismuth-207, Cadmium-109, Cadmium-15m, Calcium-45, Cerium-139, Cerium-141, Cerium-144, Cesium-137, Chromium-51, Cobalt-55, Cobalt-56, Cobalt-57, Cobalt-58, Cobalt-60, Cobalt-64, Copper-67, Erbium-169, Europium-152, Gallium-64, Gallium-68, Gadolinium-153, Gadolinium-157 Gold-195, Gold-199, Hafnium-175, Hafiium-175-181, Holmium-166, Indium-110, Indium-111, Iridium-192, Iron-55, Iron-59, Krypton-85, Lead-210, Manganese-54, Mercury-197, Mercury-203, Molybdenum-99, Neodymium-147, Neptunium-237, Nickel-63, Niobium-95, Osmium-185+191, Palladium-103, Platinum-195m, Praseodymium-143, Promethium-147, Protactinium-233, Radium-226, Rhenium-186, Rhenium-188, Rubidium-86, Ruthenium-103, Ruthenium-106, Scandium-44, Scandium-46, Selenium-75, Silver-110m, Silver-11, Sodium-22, Strontium-85, Strontium-89, Strontium-90, Sulfur-35, Tantalum-182, Technetium-99m, Tellurium-125, Tellurium-132, Thallium-204, Thorium-228, Thorium-232, Thallium-170, Tin-113, Tin-114, Tin-117m, Titanium-44, Tungsten-185, Vanadium-48, Vanadium-49, Ytterbium-169, Yttrium-86, Yttrium-88, Yttrium-90, Yttrium-91, Zinc-65, and Zirconium-95.

Non-Metallic Radionuclides

The compounds of the invention can also comprise one or more (e.g., 1, 2, 3, or 4) non-metallic radionuclide which can be directly linked to a residue of the compound of formula I at any synthetically feasible site, or can be linked to a residue of the compound of formula I, by a linker, at any synthetically feasible site. Suitable linkers are described herein. In addition, suitable points of attachment of a the compound of formula I for the non-metallic radionuclide, either directly or by a linker, are also described herein. The invention also provides compounds having more than one non-metallic radionuclide attached to a compound of formula I, either directly, or by a linker.

Specifically, the non-metallic radionuclide can be a non-metallic paramagnetic atom (e.g., Fluorine-19); or a non-metallic positron emitting radionuclide (e.g., Carbon-11, Fluorine-18, Iodine-123, or Bromine-76). Fluorine-18 is a suitable non-metallic radionuclide for use the compounds of the present invention in part because there is typically little or no background noise associated with the diagnostic use of fluorine in the body of a mammal (e.g., human). Preferably, the detectable radionuclide is a non-metallic radionuclide, e.g., Carbon-11, Fluorine-18, Bromine-76, Iodine-123, Iodine-124.

The compounds disclosed herein can be prepared using procedures similar to those described in U.S. Pat. No. 5,739,313, or using procedures similar to those described herein. The residue of a molecules comprising B-10 can be linked to the residue of a compound of formula I as described herein. Additional compounds, intermediates, and synthetic preparations thereof are disclosed, for example, in Hogenkamp, H. et al., Synthesis and Characterization of nido-Carborane-Cobalamin Conjugates, Nucl. Med. & Biol., 2000, 27, 89-92; Collins, D., et al., Tumor Imaging Via Indium 111-Labeled DTPA-Adenosylcobalamin, Mayo Clinic Proc., 1999, 74:687-691; U.S. Application Ser. No. 60/129,733 filed 16 Apr. 1999; U.S. Application Ser. No. 60/159,874 filed 15 Oct. 1999; U.S. Application Ser. No. 60/159,753 filed 15 Oct. 1999; U.S. Application Ser. No. 60/159,873 filed 15 Oct. 1999; and references cited therein.

A specific compound of the present invention is compound wherein a residue of the compound of formula I is linked directly or by a linker to a residue of a chemotherapeutic agent; wherein X is CN; or a pharmaceutically acceptable salt thereof.

Another specific compound of the present invention is compound wherein a residue of the compound of formula I is linked directly or by a linker to a residue of a chemotherapeutic agent; wherein the compound of formula I is linked directly or by a linker to a detectable radionuclide; wherein X is CN; or a pharmaceutically acceptable salt thereof.

Another specific compound of the present invention is a compound wherein a residue of a chemotherapeutic agent is linked directly or by a linker to a residue of the b-, d-, or e-carboxamide of a compound of formula I; or a pharmaceutically acceptable salt thereof.

Another specific compound of the present invention is a compound wherein a residue of a chemotherapeutic agent is linked directly or by a linker to a residue of the b-, d-, or e-carboxamide of a compound of formula I; wherein a detectable radionuclide is linked directly or by a linker to a residue of the b-, d-, or e-carboxamide of a compound of formula I; or a pharmaceutically acceptable salt thereof.

Another specific compound of the present invention is a compound wherein a residue of a compound of formula I, wherein X is CN is linked directly or by a linker to a residue of an antineoplastic agent; or a pharmaceutically acceptable salt thereof.

Another specific compound of the present invention is a compound wherein a residue of a compound of formula I, wherein X is CN is linked directly or by a linker to a residue of an antineoplastic agent; wherein a residue of a compound of formula I is linked directly or by a linker to a detectable radionuclide; or a pharmaceutically acceptable salt thereof.

Another specific compound of the present invention is a compound wherein a residue of a compound of formula I, wherein X is CN is linked directly or by a linker to a residue of paclitaxel or doxorubicin; or a pharmaceutically acceptable salt thereof.

Another specific compound of the present invention is a compound wherein a residue of a compound of formula I, wherein X is CN is linked directly or by a linker to a residue of paclitaxel or doxorubicin; wherein a residue of a compound of formula I is linked directly or by a linker to a detectable radionuclide; or a pharmaceutically acceptable salt thereof.

Another specific compound of the present invention is a compound wherein a residue of the compound of formula I is linked directly or by a linker to a residue of paclitaxel or doxorubicin at the b-, d-, or e-carboxamide; or a pharmaceutically acceptable salt thereof.

Another specific compound of the present invention is a compound wherein a residue of the compound of formula I is linked directly or by a linker to a residue of paclitaxel or doxorubicin at the b-, d-, or e-carboxamide; wherein a residue of a compound of formula I is linked directly or by a linker to a detectable radionuclide; or a pharmaceutically acceptable salt thereof.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The present invention provides a method of treating a tumor in a mammal. The tumor can be located in any part of the mammal. Specifically, the tumor can be located in the breast, lung, thyroid, lymph node, genitourinary system (e.g., kidney, ureter, bladder, ovary, teste, or prostate), musculoskeletal system (e.g., bones, skeletal muscle, or bone marrow), gastrointestinal tract (e.g., stomach, esophagus, small bowel, colon, rectum, pancreas, liver, or smooth muscle), central or peripheral nervous system (e.g., brain, spinal cord, or nerves), head and neck tumors (e.g., ears, eyes, nasopharynx, oropharynx, or salivary glands), or the heart.

The compound of the present invention (cobalamin conjugates) can be formulated as pharmaceutical compositions and administered to a mammalian host, such as a human patient in a variety of forms adapted to the chosen route of administration, i.e., orally or parenterally, by intravenous, intramuscular, or subcutaneous routes.

Thus, the cobalamin conjugates may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the substance may be combined with one or more excipients and used in the form of ingestible tablets, buccal tablets, troches, capsules, elixirs, suspensions, syrups, wafers, and the like. Such compositions and preparations should contain at least 0.1% of the substance. The percentage of the compositions and preparations may, of course, be varied and may conveniently be between about 2 to about 60% of the weight of a given unit dosage form. The amount of substance in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the substance may be incorporated into sustained-release preparations and devices.

The cobalamin conjugates can also be administered intravenously or intraperitoneally by infusion or injection. Solutions of the substance can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the substance which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, normal saline, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the substance in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the substance required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The substance is conveniently administered in unit dosage form; for example, containing 5 to 1000 mg, conveniently 10 to 750 mg, most conveniently, 50 to 500 mg of active ingredient per unit dosage form.

Ideally, the substance should be administered to achieve peak plasma concentrations of from about 0.5 to about 75 μM, preferably, about 1 to 50 μM, most preferably, about 2 to about 30 μM. This may be achieved, for example, by the intravenous injection of a 0.05 to 5% solution of the substance, optionally in saline, or orally administered as a bolus containing about 1-100 mg of the substance. Desirable blood levels may be maintained by continuous infusion to provide about 0.01-5.0 mg/kg/hr or by intermittent infusions containing about 0.4-15 mg/kg of the substance.

The substance may conveniently be presented in a single dose or as divided doses administered at appropriate intervals, for example, as two, three, four or more sub-doses per day.

The invention will now be illustrated by the following non-limiting Examples.

### EXAMPLES

### Example 1

Proposed Synthesis of Daunorubicin- and Doxorubicin-Cobalamin Conjugates

Modification of the carbohydrate moiety (daunosamine) of daunorubicin (1) with L-leucine can be accomplished by reacting daunorubicin HCl (0.5 g) in 100 mL borate buffer pH=10 (containing KCl) with L-leucine-carboxyanhydride (1 mmol in 5 mL acetone) at 0° C. under nitrogen. After reaction for 5 minutes at 0° C., the mixture can be acidified to pH 3.5 with H₂SO₄, stirred for 15 minutes and adjusted to pH=7 to give the desired L-leucyl daunorubicin (2). Reaction of (2) with a cobalamin-mono or dicarboxylic acid in the presence of a water-soluble carbodiimide and hydroxybenzotriazole will yield the daunorubicin-cobalamin conjugates (3). These conjugates can be isolated via the usual phenol extraction, extensive washing of the phenol phase with water and finally displacing the cobalamin-conjugates from the phenol phase into water by the addition of acetone and diethyl ether.

Modification of doxorubicin should be similar (Ger. Patent 1,813,518, Jul. 10, 1969; Chem Abstracts, 71, 91866 (1969)). D. Deprez-Decampaneere, M. Mosquelier, R. Bourain and A. Trosect, Curr. Chemother. Proc., Int. Congr. Chemother., 10th, p. 1242 (1978) have found that N-(L-leucyl) daunorubicin but not the D isomer was hydrolyzed in vivo to regenerate daunorubicin. See, “Doxorubicin, Anticancer Antibiotics,” Federico Arcamone, Medicinal Chemistry, Vol. 17, Academic Press, 1981.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound wherein a residue of a compound of formula I (FIG. 1) is linked directly or by a linker to a residue of one or more chemotherapeutic agents; wherein X is CN, OH, CH₃, or adenosyl; or a pharmaceutically acceptable salt thereof.

2. The compound of claim 1 wherein a residue of a chemotherapeutic agent is directly linked to a residue of the b, d or e-carboxamide of the compound of formula I.

3. The compound of claim 1 wherein a residue of a chemotherapeutic agent is linked by a linker to a residue of the b-, d- or e-carboxamide of the compound of formula I.

4. The compound of claim 1 wherein a residue of a chemotherapeutic agent is directly linked to the b-carboxamide of the compound of formula I.

5. The compound of claim 1 wherein a residue of a chemotherapeutic agent is linked by a linker to a residue of the b-carboxamide of the compound of formula I.

6. The compound of claim 1 wherein a residue of a chemotherapeutic agent is directly linked to a residue of the d-carboxamide of the compound of formula I.

7. The compound of claim 1 wherein a residue of a chemotherapeutic agent is linked by a linker to a residue of the d-carboxamide of the compound of formula I.

8. The compound of claim 1 wherein a residue of a chemotherapeutic agent is directly linked to a residue of the e-carboxamide of the compound of formula I.

9. The compound of claim 1 wherein a residue of a chemotherapeutic agent is linked by a linker to a residue of the e-carboxamide of the compound of formula I.

10. The compound of claim 1 wherein a residue of a first chemotherapeutic agent is linked directly or by a linker to a residue of the b-carboxamide of the compound of formula I and a residue of a second chemotherapeutic agent is linked directly or by a linker to a residue of the d-carboxamide of the compound of formula I.

11. The compound of claim 1 wherein a residue of a first chemotherapeutic agent is linked by a linker to a residue of the b-carboxamide of the compound of formula I and a residue of a second chemotherapeutic agent is linked by a linker to a residue of the d-carboxamide of the compound of formula I.

12. The compound of claim 1 wherein the chemotherapeutic agent is an antineoplastic agent.

13. The compound of claim 12 wherein the antineoplastic agent is a cytotoxic agent.

14. The compound of claim 13 wherein the cytotoxic agent is doxorubicin or paclitaxel.

15. The compound of any one of claims 1, 3, 5, 7, 9, 10, and 11 wherein at least one linker is of the formula W-A-Q wherein A is (C₁-C₆)alkyl, (C₂-C₆)alkenyl, (C₂-C₆)alkynyl, (C₃-C₈)cycloalkyl, or (C₆-C₁₀)aryl, wherein W and Q are each independently —N(R)C(═O)—, —C(═O)N(R)—, —OC(═O)—, —C(═O)O—, —O—, —S—, —S(O)—, —S(O)₂—, —N(R)—, —C(═O)—, or a direct bond; wherein each R is independently H or (C₁-C₆)alkyl.

16. The compound of claim 15 wherein W and Q are each —N(R)—.

17. The compound of any one of claims 1, 3, 5, 7, 9, 10, and 11 wherein at least one linker is of the formula W—(CH₂)ₙ-Q wherein, n is between about 1 and about 20, between about 1 and about 15, between about 2 and about 10, between about 2 and about 6, or between about 4 and about 6; wherein W and Q are each independently —N(R)C(═O)—, —C(═O)N(R)—, —OC(═O)—, —C(═O)O—, —O—, —S—, —S(O)—, —S(O)₂—, —C(═O)—, —N(R)—, or a direct bond; wherein each R is independently H or (C₁-C₆)alkyl.

18. The compound of claim 17 wherein at least one of W and Q is —N(R)—.

19. The compound of claim 18 wherein n is in the range from about 2 to about 6, inclusive.

20. The compound of any one of claims 1, 3, 5, 7, 9, 10, and 11 wherein the linker is a divalent radical formed from a peptide or an amino acid.

21. The compound of claim 20 wherein the peptide comprises 2 to about 25 amino acids.

22. The compound of claim 20 wherein the peptide is poly-L-lysine, containing about 8 to about 11 residues.

23. The compound of any one of claims 1, 3, 5, 7, 9, 10, and 11 wherein the linker is a 1,ω-divalent radical formed from a peptide.

24. The compound of any one of claims 1, 3, 5, 7, 9, 10, and 11 wherein the linker separates the residue of a compound of formula I from the residue of the chemotherapeutic agent by about 5 angstroms to about 50 angstroms.

25. A compound wherein a residue of a compound of formula I is linked directly or by a linker to a residue of a chemotherapeutic agent through the 6-position and wherein a residue of the compound of formula I is linked directly or by a linker to a residue of one or more additional chemotherapeutic agents; or a pharmaceutically acceptable salt thereof.

26. The compound of claim 25 wherein a residue of a chemotherapeutic agent is directly linked to a residue of the b-, d- or e-carboxamide of the compound of formula I.

27. The compound of claim 25 wherein a residue of a chemotherapeutic agent is linked by a linker to a residue of the b-, d- or e-carboxamide of the compound of formula I.

28. The compound of claim 25 wherein a residue of a chemotherapeutic agent is directly linked to the b-carboxamide of the compound of formula I.

29. The compound of claim 25 wherein a residue of a chemotherapeutic agent is linked by a linker to a residue of the b-carboxamide of the compound of formula I.

30. The compound of claim 25 wherein a residue of a chemotherapeutic agent is directly linked to a residue of the d-carboxamide of the compound of formula I.

31. The compound of claim 25 wherein a residue of a chemotherapeutic agent is linked by a linker to a residue of the d-carboxamide of the compound of formula I.

32. The compound of claim 25 wherein a residue of a chemotherapeutic agent is directly linked to a residue of the e-carboxamide of the compound of formula I.

33. The compound of claim 25 wherein a residue of a chemotherapeutic agent is linked by a linker to a residue of the e-carboxamide of the compound of formula I.

34. The compound of claim 25 wherein a residue of a chemotherapeutic agent is linked directly or by a linker to a residue of the b-carboxamide of the compound of formula I and a residue of a second chemotherapeutic agent is linked directly or by a linker to a residue of the d-carboxamide of the compound of formula I.

35. The compound of claim 25 wherein a residue of a chemotherapeutic agent is linked by a linker to a residue of the b-carboxamide of the compound of formula I and a residue of a second chemotherapeutic agent is linked by a linker to a residue of the d-carboxamide of the compound of formula I.

36. The compound of any one of claims 25, 27, 29, 31, 33, 34 and 35 wherein at least one linker is of the formula W-A-Q wherein A is (C₁-C₆)alkyl, (C₂-C₆)alkenyl, (C₂-C₆)alkynyl, (C₃-C₈)cycloalkyl, or (C₆-C₁₀)aryl, wherein W and Q are each independently —N(R)C(═O)—, —C(═O)N(R)—, —OC(═O)—, —C(═O)O—, —O—, —S—, —S(O)—, —S(O)₂—, —N(R)—, —C(═O)—, or a direct bond; wherein each R is independently H or (C₁-C₆)alkyl.

37. The compound of claim 36 wherein at least one of W and Q is —N(R)—.

38. The compound of any one of claims 25, 27, 29, 31, 33, 34 and 35 wherein at least one linker is of the formula W—(CH₂)ₙ-Q wherein, n is between about 1 and about 20, between about 1 and about 15, between about 2 and about 10, between about 2 and about 6, or between about 4 and about 6; wherein W and Q are each independently —N(R)C(═O)—, —C(═O)N(R)—, —OC(═O)—, —C(═O)O—, —O—, —S—, —S(O)—, —S(O)₂—, —C(═O)—, —N(R)—, or a direct bond; wherein each R is independently H or (C₁-C₆)alkyl.

39. The compound of claim 38 wherein at least one of W and Q is —N(R)—.

40. The compound of claim 38 wherein n is in the range of about 2 to about 6, inclusive.

41. The compound of anyone of claims 25, 27, 29, 31, 33, 34 and 35 wherein a linker is a divalent radical formed from a peptide or an amino acid.

42. The compound of claim 41 wherein the peptide comprises 2 to about 25 amino acids.

43. The compound of claim 42 wherein the peptide is poly-L-lysine, containing about 8 to about 11 residues.

44. The compound of any one of claims 25, 27, 29, 31, 33, 34 and 35 wherein the linker is a 1,ω-divalent radical formed from a peptide.

45. The compound of any one of claims 25, 27, 29, 31, 33, 34 and 35 wherein the linker separates the residue of a compound of formula I from the residue of the chemotherapeutic agent by about 5 angstroms to about 50 angstroms.

46. The compound of claim 25 wherein the chemotherapeutic agent is an antineoplastic agent.

47. The compound of claim 46 wherein the antineoplastic agent is a cytotoxic agent.

48. The compound of claim 47 wherein the cytotoxic agent is doxorubicin or paclitaxel.

49. A compound of formula II 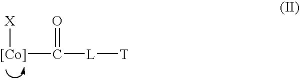
wherein 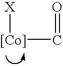
is a residue of the compound of formula I;
X is CN, OH, CH₃, adenosyl, or LL-TT wherein LL is a linker or is absent and TT is a residue of a chemotherapeutic agent;
L is a linker or absent; and
T is a residue of a chemotherapeutic agent; or a pharmaceutically acceptable salt thereof.

50. The compound of claim 49 wherein L and LL are each independently of the formula W-A-Q wherein A is (C₁-C₆)alkyl, (C₂-C₆)alkenyl, (C₂-C₆)alkynyl, (C₃-C₈)cycloalkyl, or (C₆-C₁₀)aryl, wherein W and Q are each independently —N(R)C(═O)—, —C(═O)N(R)—, —OC(═O)—, —C(═O)O—, —O—, —S—, —S(O)—, —S(O)₂—, —N(R)—, —C(═O)—, or a direct bond; wherein each R is independently H or (C₁-C₆)alkyl.

51. The compound of claim 50 wherein at least one of W and Q is —N(R)—.

52. The compound of claim 49 wherein L and LL are each independently of the formula W—(CH₂)ₙ-Q wherein, n is between about 1 and about 20, between about 1 and about 15, between about 2 and about 10, between about 2 and about 6, or between about 4 and about 6; wherein W and Q are each independently —N(R)C(═O)—, —C(═O)N(R)—, —OC(═O)—, —C(═O)O—, —O—, —S—, —S(O)—, —S(O)₂—, —C(═O)—, —N(R)—, or a direct bond; wherein each R is independently H or (C₁-C₆)alkyl.

53. The compound of claim 52 wherein at least one of W and Q is —N(R)—.

54. The compound of claim 52 wherein n is between about 2 and about 6.

55. The compound of claim 49 wherein L separates T and the residue by about 5 angstroms to about 200 angstroms.

56. The compound of claim 49 wherein at least one of L and LL is a divalent radical formed from a peptide or an amino acid.

57. The compound of claim 56 wherein the peptide comprises 2 to about 25 amino acids.

58. The compound of claim 56 wherein the peptide is poly-L-lysine, containing about 8 to about 11 residues.

59. The compound of claim 49 wherein at least one of L and LL is a 1,ω-divalent radical formed from a peptide or an amino acid.

60. The compound of claim 49 wherein at least one of T and TT is a residue of paclitaxel or doxorubicin, or a pharmaceutically acceptable salt thereof.

61. The compound of claim 49 wherein the (C═O) in the group is attached to L-T at the b-, d- or e-position.

62. A compound of formula II 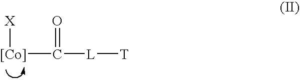
wherein 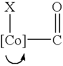
is a residue of the compound of formula I;
X is LL-TT wherein LL is a linker or is absent and TT is a residue of a chemotherapeutic agent;
L is a linker or absent; and
T is a residue of a chemotherapeutic agent; or a pharmaceutically acceptable salt thereof.

63. The compound of claim 62 wherein L and LL are each independently of the formula W-A-Q wherein A is (C₁-C₆)alkyl, (C₂-C₆)alkenyl, (C₂-C₆)alkynyl, (C₃-C₈)cycloalkyl, or (C₆-C₁₀)aryl, wherein W and Q are each independently —N(R)C(═O)—, —C(═O)N(R)—, —OC(═O)—, —C(═O)O—, —O—, —S—, —S(O)—, —S(O)₂—, —N(R)—, —C(═O)—, or a direct bond; wherein each R is independently H or (C₁-C₆)alkyl.

64. The compound of claim 63 wherein at least one of W and Q is —N(R)—.

65. The compound of claim 62 wherein L and LL are each independently of the formula W—(CH₂)ₙ-Q wherein, n is between about 1 and about 20, between about 1 and about 15, between about 2 and about 10, between about 2 and about 6, or between about 4 and about 6; wherein W and Q are each independently —N(R)C(═O)—, —C(═O)N(R)—, —OC(═O)—, —C(═O)O—, —O—, —S—, —S(O)—, —S(O)₂—, —C(═O)—, —N(R)—, or a direct bond; wherein each R is independently H or (C₁-C₆)alkyl.

66. The compound of claim 65 wherein at least one of W and Q is —N(R)—.

67. The compound of claim 65 wherein n is between about 2 and about 6.

68. The compound of claim 62 wherein L separates T and the residue by about 5 angstroms to about 200 angstroms.

69. The compound of claim 62 wherein at least one of L and LL is a divalent radical formed from a peptide or an amino acid.

70. The compound of claim 69 wherein the peptide comprises 2 to about 25 amino acids.

71. The compound of claim 69 wherein the peptide is poly-L-lysine, containing about 8 to about 11 residues.

72. The compound of claim 62 wherein at least one of L and LL is a 1,ω-divalent radical formed from a peptide.

73. The compound of claim 62 wherein at least one of T and TT is a residue of paclitaxel or doxorubicin, or a pharmaceutically acceptable salt thereof.

74. The compound of claim 62 wherein (C═O) in the group is attached to L-T is attached at the b-, d- or e-position.

75. A compound of formula III: 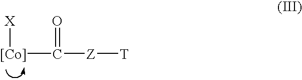
wherein 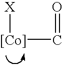
X is CN, OH, CH₃, adenosyl, or ZZ-TT wherein ZZ is a linker or is absent and TT is a residue of a chemotherapeutic agent;
Z is —N(R)—, —O—, or —S—, wherein R is H or (C₁-C₆)alkyl or absent; and
T is a residue of a chemotherapeutic agent; or a pharmaceutically acceptable salt thereof.

76. The compound of claim 75 wherein Z and ZZ are each independently of the formula W-A-Q wherein A is (C₁-C₆)alkyl, (C₂-C₆)alkenyl, (C₂-C₆)alkynyl, (C₃-C₈)cycloalkyl, or (C₆-C₁₀)aryl, wherein W and Q are each independently —N(R)C(═O)—, —C(═O)N(R)—, —OC(═O)—, —C(═O)O—, —O—, —S—, —S(O)—, —S(O)₂—, —N(R)—, —C(═O)—, or a direct bond; wherein each R is independently H or (C₁-C₆)alkyl.

77. The compound of claim 76 wherein at least one of W and Q is —N(R)—.

78. The compound of claim 75 wherein Z and ZZ are each independently of the formula W—(CH₂)ₙ-Q wherein, n is between about 1 and about 20, between about 1 and about 15, between about 2 and about 10, between about 2 and about 6, or between about 4 and about 6; wherein W and Q are each independently —N(R)C(═O)—, —C(═O)N(R)—, —OC(═O)—, —C(═O)O—, —O—, —S—, —S(O)—, —S(O)₂—, —C(═O)—, —N(R)—, or a direct bond; wherein each R is independently H or (C₁-C₆)alkyl.

79. The compound of claim 78 wherein at least one of W and Q is —N(R)—.

80. The compound of claim 78 wherein n is between about 2 and about 6.

81. The compound of claim 75 wherein Z separates T and the residue by about 5 angstroms to about 200 angstroms.

82. The compound of claim 75 wherein at least one of Z and ZZ is a divalent radical formed from a peptide or an amino acid.

83. The compound of claim 82 wherein the peptide comprises 2 to about 25 amino acids.

84. The compound of claim 75 wherein the peptide is poly-L-lysine, containing about 8 to about 11 residues.

85. The compound of claim 75 wherein at least one of Z and ZZ is a 1,ω-divalent radical formed from a peptide.

86. The compound of claim 75 wherein the (C═O) in the group is attached to Z-T at the b-, d- or e-position.

87. The compound of claim 75 wherein at least one of T and TT is a residue of an antineoplastic agent.

88. The compound of claim 87 wherein the antineoplastic agent is a cytotoxic agent.

89. The compound of claim 88 wherein the cytotoxic agent is doxorubicin or paclitaxel.

90. A compound of formula III: 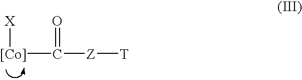
wherein 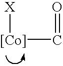
is a residue of the compound of formula I;
X is LL-TT wherein LL is a linker or is absent and TT is a residue of a chemotherapeutic agent;
Z is —N(R)—, —O—, or —S—, wherein R is H, (C₁-C₆)alkyl, or absent; and
T is a residue of a chemotherapeutic agent; or a pharmaceutically acceptable salt thereof.

91. The compound of claim 90 wherein Z and ZZ are each independently of the formula W-A-Q wherein A is (C₁-C₆)alkyl, (C₂-C₆)alkenyl, (C₂-C₆)alkynyl, (C₃-C₈)cycloalkyl, or (C₆-C₁₀)aryl, wherein W and Q are each independently —N(R)C(═O)—, —C(═O)N(R)—, —OC(═O)—, —C(═O)O—, —O—, —S—, —S(O)—, —S(O)₂—, —N(R)—, —C(═O)—, or a direct bond; wherein each R is independently H or (C₁-C₆)alkyl.

92. The compound of claim 91 wherein at least one of W and Q is —N(R)—.

93. The compound of claim 90 wherein Z and ZZ are each independently of the formula W—(CH₂)ₙ-Q wherein, n is between about 1 and about 20, between about 1 and about 15, between about 2 and about 10, between about 2 and about 6, or between about 4 and about 6; wherein W and Q are each independently —N(R)C(═O)—, —C(═O)N(R)—, —OC(═O)—, —C(═O)O—, —O—, —S—, —S(O)—, —S(O)₂—, —C(═O)—, —N(R)—, or a direct bond; wherein each R is independently H or (C₁-C₆)alkyl.

94. The compound of claim 93 wherein at least one of W and Q is —N(R)— wherein each R is independently H or (C₁-C₆)alkyl.

95. The compound of claim 93 wherein n is between about 2 and about 6.

96. The compound of claim 90 wherein Z separates T and the residue by about 5 angstroms to about 200 angstroms.

97. The compound of claim 90 wherein at least one of Z and ZZ is a divalent radical formed from a peptide or an amino acid.

98. The compound of claim 97 wherein the peptide comprises 2 to about 25 amino acids.

99. The compound of claim 90 wherein the peptide is poly-L-lysine, containing about 8 to about 11 residues.

100. The compound of claim 90 wherein at least one of Z and ZZ is a 1,ω-divalent radical formed from a peptide.

101. The compound of claim 90 wherein (C═O) in the group is attached to Z-T at the b-, d- or e-position.

102. The compound of claim 90 wherein at least one of T and TT is a residue of an antineoplastic agent.

103. The compound of claim 102 wherein the antineoplastic agent is a cytotoxic agent.

104. The compound of claim 103 wherein the cytotoxic agent is doxorubicin or paclitaxel.

105. A compound wherein a residue of a compound of formula I (FIG. 1) is linked directly or by a linker to a residue of one or more chemotherapeutic agents; wherein X is CN, OH, CH₃, or adenosyl; wherein the compound of formula I is also linked directly or by a linker to a detectable radionuclide; or a pharmaceutically acceptable salt thereof.

106. The compound of claim 105 wherein the detectable radionuclide is linked to a residue of the b, d or e-carboxamide of the compound of formula I.

107. The compound of claim 105 wherein the detectable radionuclide is linked by a linker to a residue of the compound of formula I.

108. The compound of claim 105 wherein the detectable radionuclide is directly linked to a residue of the compound of formula I.

109. The compound of claim 105 wherein the detectable radionuclide is a non-metallic radionuclide.

110. The compound of claim 109 wherein the non-metallic radionuclide is Carbon-11, Fluorine-18, Bromine-76, Iodine-123, or Iodine-124.

111. A pharmaceutical composition comprising a compound of any one of claims 1-110 and a pharmaceutically acceptable carrier.

112. A compound of any one of claims 1-110 for use in medical therapy or diagnosis.

113. The use of claim 105 for the manufacture of a medicament for imaging a tumor in a mammal.

114. The use of claim 113 wherein the tumor is located in the breast, lung, thyroid, lymph node, kidney, ureter, bladder, ovary, teste, prostate, bone, skeletal muscle, bone marrow, stomach, esophagus, small bowel, colon, rectum, pancreas, liver, smooth muscle, brain, spinal cord, nerves, ear, eye, nasopharynx, oropharynx, salivary glands, or the heart.

115. The use of a compound of any one of claims 1-110 for the manufacture of a medicament for treating a tumor in a mammal.

116. The use of claim 115 wherein the tumor is located in the breast, lung, thyroid, lymph node, kidney, ureter, bladder, ovary, teste, prostate, bone, skeletal muscle, bone marrow, stomach, esophagus, small bowel, colon, rectum, pancreas, liver, smooth muscle, brain, spinal cord, nerves, ear, eye, nasopharynx, oropharynx, salivary glands, or the heart.

117. A method of treating a tumor in a mammal in need of such treatment comprising administering to the mammal an effective amount of a compound of any one of claims 1-110.

118. The method of claim 117 wherein the tumor is located in the breast, lung, thyroid, lymph node, kidney, ureter, bladder, ovary, teste, prostate, bone, skeletal muscle, bone marrow, stomach, esophagus, small bowel, colon, rectum, pancreas, liver, smooth muscle, brain, spinal cord, nerves, ear, eye, nasopharynx, oropharynx, salivary glands, or the heart.

119. A method of imaging a tumor in a mammal in need of such imaging comprising administering to the mammal an effective amount of a compound of claim 105; and detecting the presence of the compound.

120. The method of claim 119 wherein the tumor is located in the breast, lung, thyroid, lymph node, kidney, ureter, bladder, ovary, teste, prostate, bone, skeletal muscle, bone marrow, stomach, esophagus, small bowel, colon, rectum, pancreas, liver, smooth muscle, brain, spinal cord, nerves, ear, eye, nasopharynx, oropharynx, salivary glands, or the heart.
